(19) United States
(12) Patent Application Publication
(10) Pub. No.: US 2004/0185299 A1
(43) Pub. Date: Sep. 23, 2004

(54) Tertiary diamines containing heterocyclic groups and their use in organic electroluminescent devices

(76) Inventor: Tuan Quoc Ly, Littlemore Oxfordshire (GB)

Correspondence Address: Marshall Gerstein & Borun, 6300 Sears Tower, 233 South Wacker Drive, Chicago, IL, 60606-6357, US

(21) Appl. No.: 10/483,802

(22) PCT Filed: Jul. 4, 2002
(86) PCT No.: PCT/GB02/03115

(30) Foreign Application Priority Data
Jul. 17, 2001 (GB) 0117377.2

(51) Int. Cl. H05B 33/12; C07D 311/02; C 07D 3 7/77

(52) U.S. Cl. 428/690; 428/917; 313/504; 313/506; 549/200; 564/427; 564/428

(57) ABSTRACT

Tertiary diamines having formula (I), wherein Ar is an aromatic group selected from: formula (n), where n=1 to 3; formula (m), where m=1 to 3; formula (p), where p=1 to 3, R¹ is a group selected from alkyl, alkenyl, cycloalkyl, cycloalkenyl, carbocyclic aryl optionally substituted by at least one group selected from halo, alkyl, cyano, nitro and cycloalkyl and an aromatic heterocyclic group optionally substituted by at least one group selected from halo, cyano, nitro, alkyl, cycloalkyl and aryl optionally substituted by at least one halo group; R² is a fused bicyclic or tricyclic aromatic heterocyclic group selected from formula (A) and formula (B), which heterocyclic group may, optionally, be substituted by at least one group selected from halo, cyano, nitro, alkyl, cycloalkyl and aryl optionally substituted by at least one halo and wherein Q is O, S or N—R⁵ where R⁵ is H, alkyl, cycloalkyl or aryl optionally substituted by at least one group selected from halo, alkyl, cyano or nitro. R³ is a group selected from alkyl, alkenyl, cycloalkyl, cycloalkenyl, carbocyclic aryl optionally substituted by at least one group selected from halo, alkyl, cyano, nitro and cycloalkyl, and an aromatic heterocyclic group optionally substituted by at least one group selected from halo, cyano, nitro, alkyl, cycloalkyl and aryl optionally substituted by at least one halo; and R⁴ is a group selected from carbocyclic aryl optionally substituted by at least one group selected from halo, cyano, nitro and alkyl, and aromatic heterocyclic optionally substituted by at least one group selected from halo, cyano, nitro, alkyl, aryl optionally substituted by at least one halo, and cycloalkyl are disclosed. These compounds are useful as light emitting materials, hole injection materials or hole transporting materials in organic light emitting devices, particularly having application in flat panel displays.

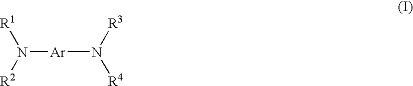

This invention relates to tertiary diamines containing heterocyclic groups, to organic electroluminescent devices incorporating them and to the use of such diamines as light emitting materials, hole injecting materials or hole transporting materials in such devices. These devices may be utilised in flat-panel displays.

Flat-panel displays are the critical enabling technology for many current applications such as mobile and video telephones and lap-top and palm-top computers.

Currently, the flat-panel display market is dominated by liquid crystal technology. However, liquid crystal display devices suffer several drawbacks such as small operational viewing angles, poor image contrast and high power consumption. As an alternative technology for flat panel displays, organic electroluminescent (also known as organic light emitting diode) displays using organic or organometallic molecules or semi-conducting polymers offer the potential for lower cost, improved viewing angles, better contrast and lower power consumption. Although organic electroluminescent displays have recently entered commercial production, there is still significant scope for enhancing performance parameters such as lifetime, efficiency and colour.

Typically, a flat-panel device comprises a multi-layer assembly of structurally important films. In such a device an electroluminescent medium is sandwiched between two electrodes, at least one which is transparent. The electroluminescent medium emits light in response to the application of an electrical potential difference across the electrodes. When the display incorporates patterned red, green and blue light emitters it can produce a colour image.

The electroluminescent medium lying between the electrodes may itself comprise separate zones, e.g., a hole injecting and transporting zone and a luminescent electron injecting and transporting zone. The interface of these two organic zones constitutes an internal junction which allows the injection of holes into the luminescent electron injecting and transporting zone, so that recombination of holes and electrons can take place giving rise to luminescence, but which blocks electron injection into the hole injecting and transporting zone. Alternatively, there may be a luminescent hole injecting and transporting zone combined with an electron injecting and transporting zone or a three layer device with separate hole injecting and transporting zone, a luminescent zone and an electron injecting and transporting zone. It is also possible for the hole injecting zone to be separate from the hole transporting zone.

In order to achieve a good charge balance in organic electroluminescent devices charge transport layers are included. The resulting devices which comprise a multi-layered structure generally exhibit improved performance compared to single layer devices which comprise an emitting material located between the electrodes of the device. In addition to high luminoefficiency the organic electroluminescent material sandwiched between the electrodes should exhibit thermal stability and operational durability if the device is to be useful in flat-panel displays. Therefore, the organic electroluminescent material needs to comprise compounds which perform well as charge transporters at higher temperatures as well as ones which meet the requirements for emission performance. The most common existing hole transporting material in conventional technology is (N,N′-di(1-naphthyl)-N,N′-diphenyl-[I,I′-biphenyl]-4,4′-diamine (NPB). The use of this material is disclosed in U.S. Pat. No. 5,061,569. Unfortunately, the glass transition temperature (Tg) of NPB is only 96° C. Because NPB has such a low Tg its application has to be restricted to devices which operate at relatively low temperatures. Furthermore, displays comprising devices containing NPB have a limited lifetime.

The present invention provides compounds which have good hole transporting, hole injection and emitting properties which are able to perform at relatively high temperatures.

According to the present invention there is provided a compound having the formula I

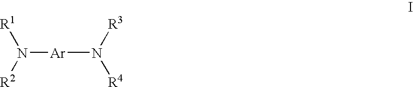

wherein Ar is an aromatic group selected from:

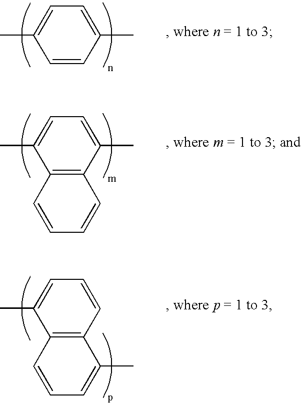

R¹ is a group selected from alkyl, alkenyl, cycloalkyl, cycloalkenyl, carbocyclic aryl optionally substituted by at least one group selected from halo, alkyl, cyano, nitro and cycloalkyl and an aromatic heterocyclic group optionally substituted by at least one group selected from halo, cyano, nitro, alkyl, cycloalkyl and aryl optionally substituted by at least one halo group;

R² is a fused bicyclic or tricyclic aromatic heterocyclic group selected from

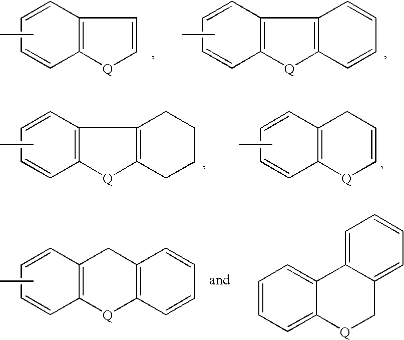

which heterocyclic group may, optionally, be substituted by at least one group selected from halo, cyano, nitro, alkyl, cycloalkyl and aryl optionally substituted by at least one halo and wherein Q is O, S or N—R⁵ where R⁵ is H, alkyl, cycloalkyl or aryl optionally substituted by at least one group selected from halo, alkyl, cyano or nitro.

R³ is a group selected from alkyl, alkenyl, cycloalkyl, cycloalkenyl, carbocyclic aryl optionally substituted by at least one group selected from halo, alkyl, cyano, nitro and cycloalkyl, and an aromatic heterocyclic group optionally substituted by at least one group selected from halo, cyano, nitro, alkyl, cycloalkyl and aryl optionally substituted by at least one halo; and

R⁴ is a group selected from carbocyclic aryl optionally substituted by at least one group selected from halo, cyano, nitro and alkyl, and aromatic heterocyclic optionally substituted by at least one group selected from halo, cyano, nitro, alkyl, aryl optionally substituted by at least one halo, and cycloalkyl.

Further according to the present invention there is provided an electroluminescent device comprising a compound of the formula I, as defined above.

Compounds of the invention have higher Tg values than NPB. They have good hole transporting properties and can be used as either hole injecting or hole transporting layers in an organic light emitting device.

In the compounds of formula I above R¹ is a group selected from alkyl, alkenyl, cycloalkyl, cycloalkenyl, carbocyclic aryl optionally substituted by at least one group selected from alkyl, halo, cyano, nitro and cycloalkyl, and an aromatic heterocyclic group optionally substituted by at least one group selected from alkyl, halo, cycloalkyl, cyano, nitro and aryl optionally substituted by at least one halo group. Preferred groups for the group R¹ are 1 to 6C alkyl, 2 to 6C alkenyl, 5 or 6C cycloalkyl, 5 or 6C cycloalkenyl, 6 to 15C aryl which may be substituted by at least one 1 to 6C alkyl, halo (e.g., F, Cl, Br and I), cyano, nitro or 5 or 6C cycloalkyl and aromatic heterocyclic groups selected from mono, bi or tricyclic heterocyclic groups containing at least one ring heteroatom selected from O, S and N, which aromatic heterocyclic group may be substituted by one or more 1 to 6C alkyl, halo (e.g., F, Cl, Br and I), 5 or 6C cycloalkyl, cyano, nitro or phenyl optionally substituted by at least one halo group. Examples of groups which are particularly preferred for R¹ include the alkyl groups methyl, ethyl, propyl, isopropyl, n-butyl, isobutyl and tert-butyl, the aromatic groups phenyl, naphthyl, arthryl, phenanthryl and pyrenyl which are optionally, but preferably, substituted by 1-6C alkyl or an electron withdrawing group selected from F, —CN and —NO₂, and the heterocyclic groups pyridyl and quinolyl which are optionally substituted by 1-6C alkyl or an electron withdrawing group selected from F, —CN or —NO₂.

In the formula I above the group R² is a fused bicyclic or tricyclic aromatic heterocyclic group selected from

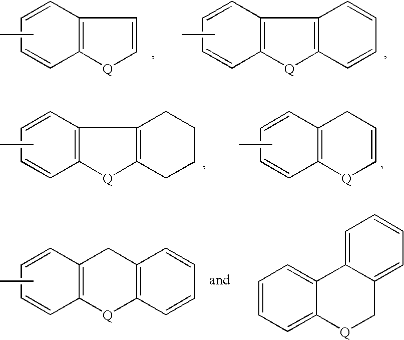

which heterocyclic group may, optionally, be substituted by at least one group selected from halo, cyano, nitro, alkyl, cycloalkyl and aryl optionally substituted by at least one halo and wherein Q is O, S or N—R⁵ where R⁵ is H, alkyl, cycloalkyl or aryl optionally substituted by at least one group selected from halo, alkyl, cyano or nitro.

Preferably, R² is a group selected from

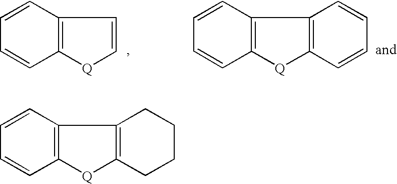

which heterocyclic group may, optionally, be substituted as described above and wherein Q is O, S or N—R⁵ where R⁵ is H, alkyl, cycloalkyl or aryl optionally substituted by at least one group selected from alkyl, halo, cyano and nitro. Examples of such heterocyclic aromatic groups include radicals derived from benzothiophene, benzofuran, indole, carbazole, chromene and xanthene. Especially preferred R² groups are carbazolyl groups of the formula

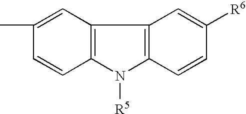

wherein R⁵ is as defined above and R⁶ is H, halo (i.e., F, Cl, Br, I), cyano, nitro, alkyl, cycloalkyl or aryl optionally substituted by at least one halo group. Such a carbazolyl group attached to the tertiary amine in the compound of formula I not only increases the Tg of the compound but, because a carbazolyl group is an electron donor to the tertiary amine group to which it is attached, it also has the effect of increasing the electron density at the tertiary amine group. Thus, a compound of the formula I having a carbazolyl group directly attached to a tertiary amine group has improved hole injection properties compared to the prior art compound NPB.

In the compound of the formula I above the group R³ is selected from alkyl, alkenyl, cycloalkyl, cycloalkenyl, carbocyclic aryl optionally substituted by at least one group selected from halo, nitro, alkyl and cycloalkyl, and an aromatic heterocyclic group optionally substituted by at least one group selected from halo, nitro, alkyl, cycloalkyl and aryl optionally substituted by at least one halo group. For examples of preferred R³ groups reference may be made to the list of groups provided above for R¹. The identity of the group R³ may be the same as or different from the identity of the group R¹. According to a preferred embodiment of the invention the groups R¹ and R³ are identical.

The group R⁴ in the formula I is selected from carbocyclic aryl groups optionally substituted by at least one group selected from halo, cyano, nitro and alkyl and aromatic heterocyclic groups optionally substituted by at least one group selected from halo, nitro, alkyl, cycloalkyl and aryl optionally substituted by at least one halo group. In the case where R⁴ is a carbocyclic aryl group optionally substituted as described above it preferably will be a 6-15C aryl optionally substituted by at least one 1 to 6C alkyl, halo (i.e., F, Cl, Br, I), cyano, nitro or 5 or 6C cycloalkyl group. Examples of such aryl groups include phenyl, naphthyl, anthryl, phenanthryl and pyrenyl any of which may be substituted by a 1-6C alkyl group or an electron withdrawing group selected from F, —CN and —NO₂. According to a preferred embodiment the group R⁴ is a fused bicyclic or tricyclic aromatic heterocyclic group containing at least one ring heteroatom selected from N, O and S which heterocyclic group is optionally substituted by at least one group selected from halo (i.e. F, Cl, Br or I), cyano, nitro, alkyl, cycloalkyl and aryl which may, itself, be substituted by at least one halo group. Preferably, R⁴ is an aromatic heterocyclic group, which is optionally substituted as described above, selected from

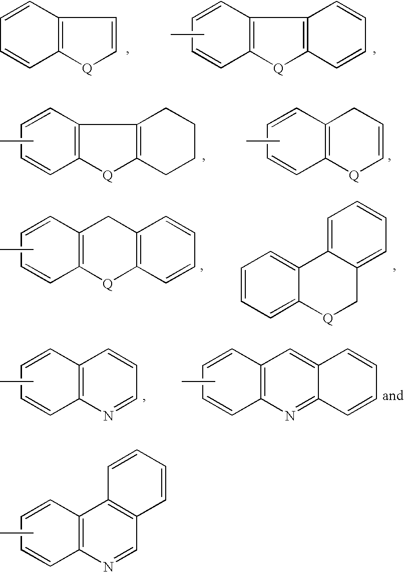

in which Q is as described above.

Examples of such groups include radicals derived from benzothiophene, benzofuran, indole, carbazole, acridine, quinoline, phenanthridine, chromene and xanthene. Preferably, the group R⁴ is identical to the group R². Especially preferred for R⁴ are carbazolyl groups as described above in connection with the discussion of R² group. It is particularly preferred that the groups R² and R⁴ are identical carbazolyl groups in view of the effects the carbazolyl groups have on the hole transporting properties of the compound, as mentioned above.

In the compound of the formula I the linking group Ar between the two tertiary amine groups is an aromatic group selected from

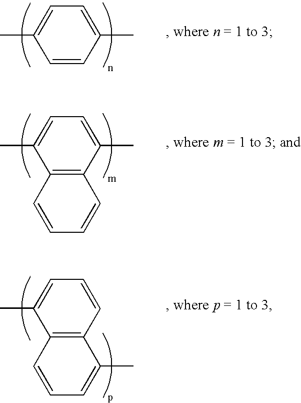

Preferably, Ar is the biphenylyl group.

Examples of compounds of the invention include N,N′-bis(9-ethylcarbazol-3-yl)-N,N′-diphenylbenzidine which is a blue emitting compound, N,N′-bis(9-ethylcarbazol-3-yl)-N,N′-di(1-naphthyl)benzidine which is a cyan emitting compound and N,N′-bis(9-ethylcarbazol-3-yl)-N,N′-di(6-quinolyl)benzidine which is a green emitting compound.

The compounds of the formula I in which R¹=R³ and R²=R⁴ can be prepared by reacting the compound

Br—Ar—Br

where Ar is as defined above with the compound R¹—NH₂, where R¹ is as defined above, to give the disubstituted secondary amine

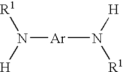

and then reacting this secondary amine with the compound R²—X, where R² is as defined above and X is halogen to give the compound

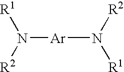

According to a preferred embodiment of the method of preparation two moles of R¹NH₂ and one mole of Br—Ar—Br are refluxed in an anhydrous aromatic solvent for several hours in the presence of palladium acetate, tri-tert-butyl phosphine and sodium tert-butoxide. The product secondary amine is then refluxed for several hours with two equivalents of R²—X also in an anhydrous aromatic solvent and also in the presence of palladium acetate, tri(t-butyl)phosphine and sodium tert-butoxide to give the desired tertiary amine. The aromatic solvent used in the coupling reaction between the aromatic halide and the amine may, for instance, be toluene or deuterated benzene, as is described by F. E. Goodson et al., J. Am. Chem. Soc. 1999, 121, 7527-7539, or o-xylene, as is described by M. Watanabe et al., Tetrahedron Letters 41 (2000) 481-483.

Asymmetrical triarylamines of the formula I above, wherein the group R¹≠R³ and/or R²≠R⁴ may be prepared by reacting the compound Br—Ar—I with the secondary amine R¹ R² NH in the presence of a copper catalyst, such as Cu Cl to give the compound

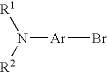

and then reacting this compound, in the presence of palladium acetate catalyst, with R³ R⁴ NH, as described above.

The selective condensation reaction of an aryl iodide and an aryl amine using a copper catalyst is described by H. B. Goodbrand et al., J. Org. Chem. 1999, 64, 670-674.

The compounds of the formula I have hole transporting properties which make them potentially useful in organic light emitting devices. The compound can be used as either hole injecting or hole transporting layers in such devices, or as the emitting layer or as a component of the emitting layer in such devices.

In general, an organic electroluminescent device comprises an anode and a cathode separated from each other by an organic luminescent material. The organic luminescent material, in its simplest form, comprises a hole injecting and transporting zone adjacent to the anode and an electron injecting and transporting zone adjacent to the cathode. More usually, however, the organic luminescent material will comprise several layers or zones, each performing as is well known in the art a different function from its neighbouring zone. In this respect, reference is made to U.S. Pat. No. 5,061,569. The compounds of the present invention have utility, in such devices, in a hole transporting zone and/or a hole injection zone as mentioned above.

### EXAMPLE 1

Preparation of N,N′-bis(9-ethylcarbazol-3-yl)-N,N′-diphenylbenzidine (Code ‘TLB1)

A reaction mixture of 3-iodo-N-ethylcarbazole (3.8 g, 11.9 mmol), N,N′-diphenylbenzidine (2 g, 5.9 mmol), sodium tertbutoxide (1.4 g, 14.3 mmol), palladium acetate (27 mg, 0.1 mmol), tritertbutyl phosphine (72 mg, 0.3 mmol) in anhydrous toluene (60 ml) was heated at reflux for 3 hours. The reaction mixture was cooled to ambient, and then hexane (100 ml) was added and finally the mixture was stirred for 2 hours. The yellow precipitate was filtered off, washed with water (200 ml) followed by hexane (100 m), and finally suction dried for 3 hours to give 4.3 g (100%) of crude product. The product was purified by sublimination at 340° C. and 1×10⁻⁷ mbar to give 2.8 g (66%) as bright yellow crystalline solid. ¹H NMR (300 MHz, CDCl₃): δ7.95 (2H, d, Ar), 7.40 (12H, m, Ar), 7.16 (8H, m, Ar), 4.37 (4H, broad q, CH₂) 1.45 (6H, t, CH₃), MS (FAB): 722 (M⁺). Found 86.5% C, 5.9% H, 7.8% N. Calc. 86.4% C, 5.9% H, 7.8% N for C₅₂H₄₂N₄. DSC: Mp=250-252° C.; Tg=136° C. TGA: decomp>450° C.

### EXAMPLE 2

Preparation of N,N′-bis(9-ethylcarbazol-3-yl)-N,N′-di(1-naphthyl)benzidine (Code ‘TLB2’)

A reaction mixture of 3-iodo-N-ethylcarbazole (3.7 g, 11.4 mmol), N,N′-di(1-naphthyl)benzidine (2.5 g, 5.7 mmol), sodium tertbutoxide (1.2 g, 12.5 mmol), palladium acetate (25 mg, 0.1 mmol), tritertbutyl phosphine (70 mg, 0.3 mmol) in anhydrous toluene (60 ml) was heated at reflux for 3 hours. The reaction mixture was cooled to ambient and then filtered, washed with toluene (50 ml) and the filtrate was saturated with hexane (300 ml) to crash out the product. The yellow precipitate was filtered off, washed with hexane (200 ml) and finally suction dried for 3 hours to give 11.2 g of crude product. The product was purified by sublimination at 360° C. and 1×10⁻⁷ mbar to give 3.7 g (79%) as bright yellow amorphous material. ¹H NMR (500 MHz, CDCl₃): δ8.13 (2H, d, Ar), 7.92 (4H, d-d, Ar), 7.74 (2H, b-s, Ar), 7.47 (8H, q, Ar), 7.43 (2H, d, Ar), 7.37 (12H, m, Ar), 7.31 (2H, d, Ar), 7.14 (2H, t, Ar), 6.90 (2H, b-s, Ar), 4.36 (4H, broad q, CH₂), 1.45 (6H, t, CH₃), MS (FAB): 822 (M⁺). Found 87.5% C, 5.6% H, 6.9% N. Calc. 87.6% C, 5.6% H, 6.8% N for C₆₀H₄₆N₄. Mp: NA for amorphous; Tg=173° C. TGA: decomp>450° C.

### EXAMPLE 3

Preparation of N,N′-Bis(9-ethylcarbazol-3-yl)-N,N′-di(6-quinolinyl)benzidine (Code ‘TLG1’)

A reaction mixture of 3-iodo-N-ethylcarbazole (2.0 g, 6.4 mmol), N,N′-di(6-quinolinyl)benzidine (1.4 g, 3.2 mmol), sodium tertbutoxide (0.7 g, 7.3 mmol), palladium acetate (14 mg, 62 μmol), triterbutyl phosphine (40 mg, 0.2 mmol) in anhydrous toluene (50 ml) was heated at reflux for 19 hours. The reaction mixture was cooled to ambient temperature and then saturated with hexane (200 ml) to crash out the product. The yellow precipitate was filtered off, washed with water (50 ml) followed by hexane (200 ml) and finally suction dried for 5 hours to give 2.3 g of crude product. The product was purified by sublimation at 430° C. and 1×10⁻⁷ mbar to give 1.6 g (61%) as bright yellow amorphous material. ¹H NMR (300 MHz, CDCl₃): δ8.70 (2H, d-d, Ar), 7.94 (6H, d-d, Ar), 7.83 (2H, d, Ar), 7.60 (2H, d-d, Ar), 7.49 (4H, d, Ar), 7.45 (2H, d, Ar), 7.40 (4H, d, Ar), 7.31 (10H, m, Ar), 7.17 (2H, t, Ar), 4.37 (4H, q, CH₂), 1.46 (6H, t, CH₃), MS (EI): 825 (M⁺). Found 84.3% C, 5.4% H, 10.2% N. Calc. 84.4% C, 5.4% H, 10.2% N for C₅₈H₄₄N₆. Mp: NA for amorphous. Tg=173° C., TGA: decomp>400° C.

### Experimental

Device Fabrication and Testing—General Procedure

Indium tin oxide (ITO) coated glass substrates, which can be purchased from several suppliers, for example Applied Films, USA or Merck Display Technology, Taiwan, are cleaned and patterned using a standard detergent and standard photolithography processes, The substrates used in the following examples measured 4″×4″ and 0.7 mm thick, the ITO was 120 nm thick, and the ITO is patterned to produce 4 devices on each substrate each with an active light emitting area of 7.4 cm². After the final stage of the photolithography process, i.e., the removal of the photoresist, the substrates are cleaned in a detergent (3 vol. % Decon 90), thoroughly rinsed in deionised water, dried and baked at 105° C. until required. Immediately prior to the formation of the device the treated substrate is oxidised in an oxygen plasma etcher. By way of example an Emitech K1050X plasma etcher operated at 100 Watts for two minutes is adequate. The substrate and shadow mask is then immediately transferred to a vacuum deposition system where the pressure is reduced to below 10⁻⁶ mbar. The organic layers are evaporated at rates between 0.5-1.5 Å/s. Then the mask is changed to form a cathode with a connection pad and no direct shorting routes. The cathode is deposited by evaporating 1.5 nm of LiF at a rate of 0.2 Å/s followed by 150 nm of aluminium evaporated at a rate of 2 Å/s.

Some devices were encapsulated at this stage using an epoxy gasket around the edge of the emissive area and a metal lid. This procedure was carried out in dry nitrogen atmosphere. The epoxy was a UV curing epoxy from Nagase, Japan.

Current/Voltage, Brightness/Voltage measurements were performed using a Keithley 2400 Source measure unit and a calibrated photodiode through a Keithley multimeter programmed from an IBM comparable PC. The EL emission spectrum was measured using an Oriel ccd camera.

Temperature Dependence of PL Emission from Devices

The photoluminescence (PL) measurements were carried out using a CCD spectrograph for light detection while excitation was provided by a UV lamp at 365 nm. The devices were prepared using the standard method described above. The structures of the devices were as follows:

Device 1: ITO/NPD/Alq₃/LiF/Al

Device 2: ITL/TLB1/Alq₃/LiF/Al

ITO—indium tin oxide

NPD—N,N′-di(1-naphthyl)-N,N′-diphenyl-[1,1′-biphenyl]4,4′-diamine

TLB1—N,N′-bis(9-ethylcarbazol-3-yl)-N,N′-diphenylbenzidine

Alq₃—tris(8-quinolinato)aluminium

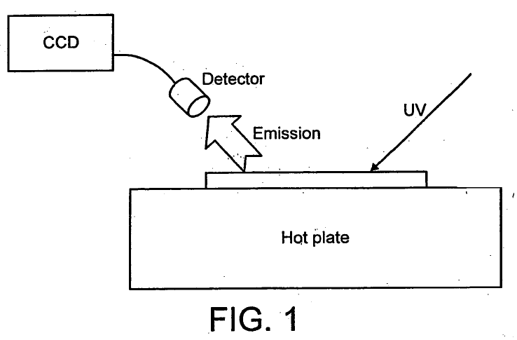

The devices were excited and the emitted light measured thorough the glass substrate. The device was positioned in such a way to avoid direct reflection of the UV light onto the detector. The device was placed on top of a hot plate that was used to vary the temperature of the device. A schematic diagram of the experimental set-up is shown in FIG. 1.

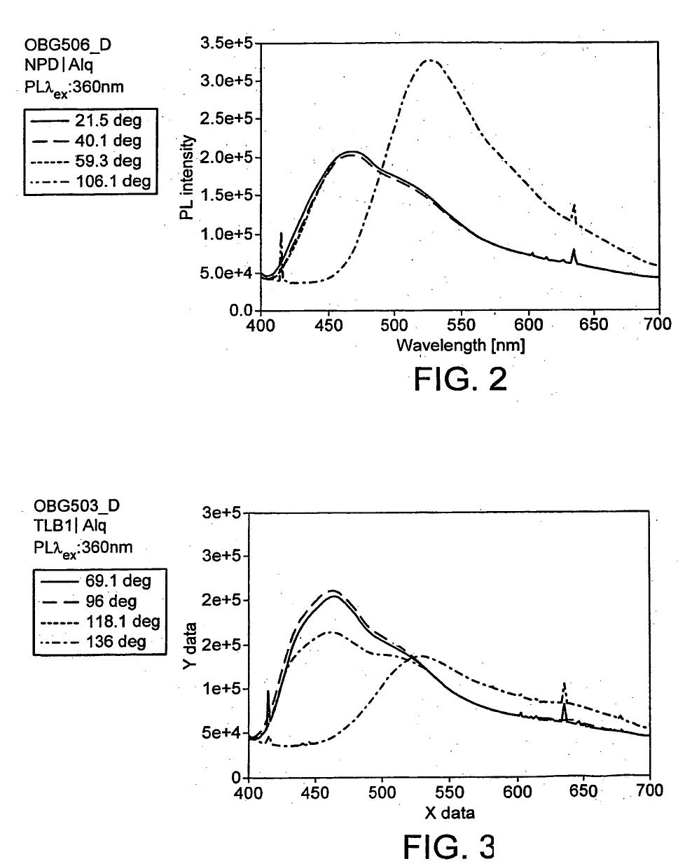

FIG. 2 shows the PL spectra of device 1 measured at different temperatures. The first three spectra, measured at 21°, 40° and 59° C., appear to be identical with emission emanating from both NPD (peak emission) and Alq₃ (shoulder emission at low energy). This is expected as the UV radiation excites the first layer (NPD), while a proportion of it is not absorbed but transmitted to Alq₃ which in turns absorbs a fraction of the light and emits. The metal cathode will reflect any remaining UV light and further absorption and emission can occur as the reflected UV light travels back through the device. The PL spectrum measured at 106° C. shows a different emission profile to the PL spectra measured at lower temperatures. The spectrum at 106° C. consists of only Alq₃ emission; the NPD emission has completely disappeared.

The same behaviour was observed when the PL of device 2 was studied as a function of temperature (see FIG. 3). At room temperature the emission profile consists of emission from TLB1 (peak emission at approximately 460 nm) and a shoulder at lower energy due to Alq₃ emission. The PL spectrum was then measured at 69°, 96°, 118° C. and showed no temperature dependence up to 118° C. However at 136° C. the emission spectrum shows only contribution from Alq₃.

The emission of device 1 (device 2), as previously explained, is expected to show contribution of both hole transporting layer, HTL, and Alq₃. This is true up to certain temperatures where both materials are thermally stable. In device 1 (device 2) the organic material of the lowest Tg is NPD (TLB1). Hence the thermal instability of the device is expected to be in the range of the Tg of the HTL. This is reflected in the emission spectra of both device 1 and device 2 as major change in their profiles occur in the range 59-106° C. and 118-136° C. respectively. Note that the Tg of NPD and TLB1 are 96° C. and 136° C., respectively. It is believed that at temperatures around the Tg of the HTL, the HTL material starts diffusing into the Alq₃ layer forming a blend where molecules of the HTL can be very close to those of Alq₃. When the device is excited using UV light, both molecules absorb light. However because of the small distance between the different molecules an efficient energy transfer from NPD's excited states to the lower lying energy states of Alq₃ occurs giving emission only from Alq₃.

TLB1, TLB2 and TLG1 Device Results

Devices were made using the compounds TLB1, TLB2 and TLG1 and were tested in order to evaluate the emission characteristics and also the hole-injection and hole-transporting properties of the materials.

Emission Characteristics TLB1:

Device structure: ITO/CuPc/TLB1/BCP/Alq/LiF/Al, where TLB1 gives blue emission, where CuPc (phthalocyanine) acts as a hole-injection layer and where BCP acts as a hole-blocking layer so that hole and electron recombination occurs principally on the TLB1 layer. The thicknesses of the layers are as follows:

CuPc: 130 Å;

TLB1: 300 Å;

BCP: 150 Å;

Alq: 200 Å;

LiF: 15 Å

Al: 1500 Å

The CIE co-ordinates from this TLB1 device are approximately (0.15, 0.14) and the peak in the emission spectrum is at approximately 442 nm. However, the spectrum shows a ‘shoulder’ of significant emission at wavelengths higher than the peak, which results in emission over a wavelength range. This feature is due to exciplex formation with the neighbouring BCP layer. TLB2 has bulkier substituents and therefore shows reduced exciplex formation; results for TLB2 emission devices are given below.

Emission Characteristics TLB2:

Device structure ITO/TLB2/BCP/Alq/LiF/Al, where TLB2 gives sky-blue emission (BCP acts as a hole-blocking layer). The thicknesses of the layers are as follows:

TLB2: 500 Å;

BCP: 150 Å;

Alq: 200 Å;

LiF: 15 Å;

Al: 1800 Å

The CIE co-ordinates from this TLB2 device are approximately (0.16, 0.29) and the peak in the emission spectrum is at approximately 486 nm.

However, a device with a hole-injection layer between the ITO and TLB2 layers was found to be more efficient. An example of such a device structure is ITO/CuPc/TLB2/BCP/Alq/LiF/Al, where the thicknesses of the layers are as follows:

CuPc: 200 Å;

TLB2: 200 Å;

BCP: 150 Å;

Alq: 200 Å;

LiF: 15 Å;

Al: 1600 Å

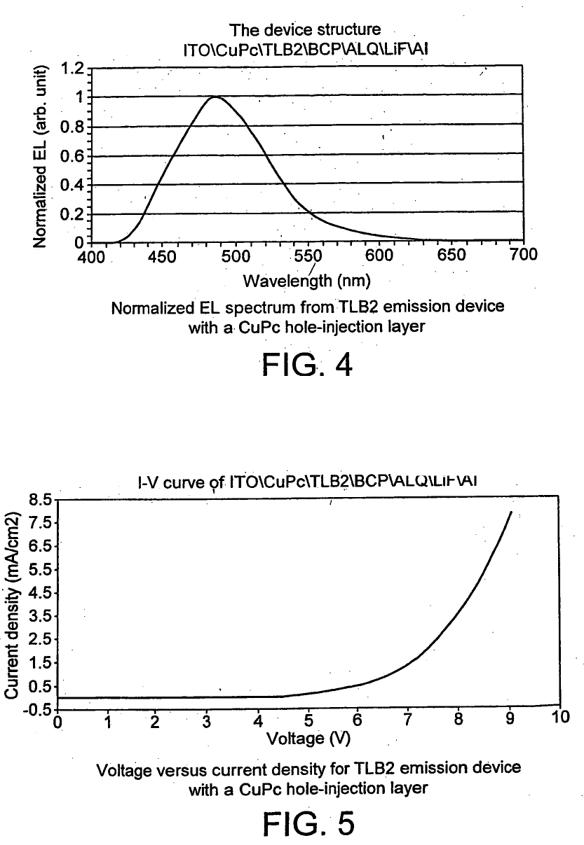

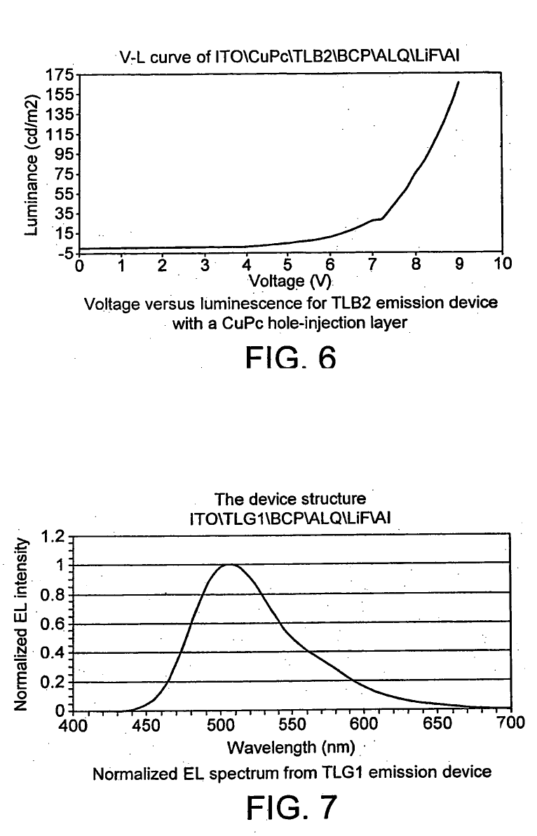

The CIE co-ordinates from this TLB2 device with a hole-injection layer are approximately (0.15, 0.30) and the peak in the emission spectrum is at approximately 488 nm. The normalised EL spectrum from the TLB2 emission device with a CuPc hole-injection layer is shown in FIG. 4. The current density of this device as a function of the supply voltage is shown in FIG. 5. The luminance of the device as a function of the supply voltage is shown in FIG. 6.

Emission Characteristics TLG1:

Device structure: ITO/TLG1/BCP/Alq/LiF/Al, where TLG1 gives green emission (BCP acting again as a hole-blocking layer). The thicknesses of the layers are as follows:

TLG1: 340 Å;

BCP: 150 Å;

Alq: 200 Å;

LiF: 15 Å;

Al: 1500 Å

The CIE co-ordinates from this TLG1 device are approximately (0.25, 0.53) and the peak in the emission spectrum is at approximately 509 nm. The normalised EL spectrum from the TLG1 emission device is shown in FIG. 7.

Hole-Injection Properties of TLB2:

Device structure: ITO/TLB2/NPB/Alq/LiF/Al, where TLB2 acts as a hole-injection layer. The thicknesses of the layers are as follows:

TLB2: 400 Å;

BCP: 70 Å

Alq: 500 Å

LiF: 15 Å

Al: 1500 Å

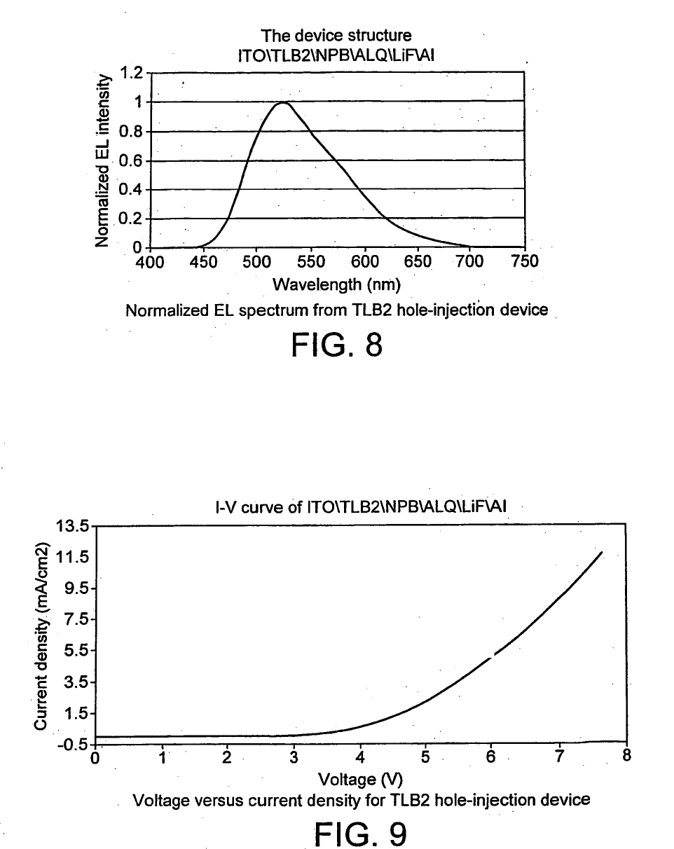

The CIE co-ordinates from this TLB2 device in which the emission is principally due to the Alq layer are approximately (0.32, 0.56) and the peak in the emission spectrum is at approximately 522 nm. The normalised EL spectrum from the TLB2 hole-injection device is shown in FIG. 8.

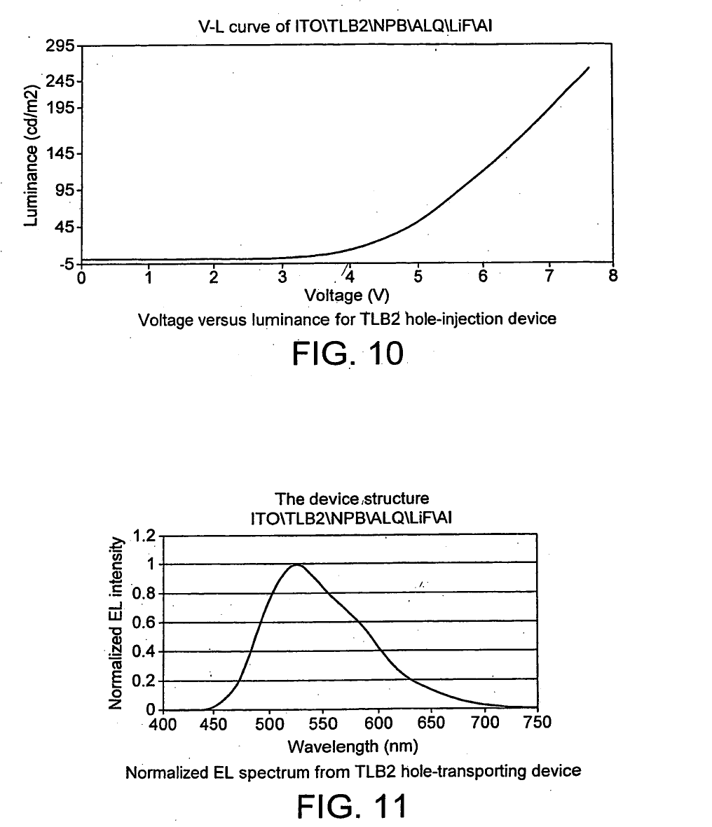

FIGS. 9 and 10, respectively, show the current density and luminance of this device as functions of the supply voltage. This device is more efficient than the non-optimized TLB2 emission device described above.

Hole-Transporting Properties of TLB2:

Device structure: ITO/TLB2/Alq/LiF/Al, where TLB2 acts as a hole-transporting layer. The thicknesses of the layers are as follows:

TLB2: 500 Å

Alq: 500 Å

LiF: 15 Å;

Al: 1500 Å

The CIE coordinates from this TLB2 device in which the emission is principally due to the Alq layer are approximately (0.33, 0.55) and the peak in the emission spectrum is at approximately 525 nm, i.e. both similar to the TLB2 hole-injection device described above, as is the EL spectrum which is shown in FIG. 11.

Comparison Between TLB1 and MTDATA:

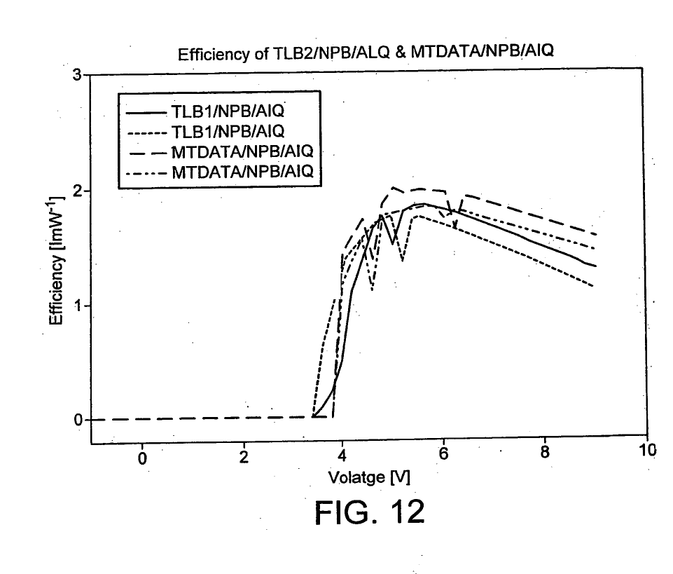

FIG. 12 shows a comparison between the hole-injecton properties of TLB1 and MTDATA. Two devices of each structure are shown, i.e. two ITO/TLB1/NBP/Alq/LiF/Al and two ITO/MTDATA/NBP/Alq/LiF/Al.

MTDATA (4, 4′, 4″-tris[3-methylphenyl(phenyl)amino]triphenylamine) is a compound that is commonly used as a good hole injection layer, placed between the ITO and NPB. However, MTDATA has a very low Tg of about 65° C. and, thus, is not suitable for commercial products. From the comparison shown in FIG. 12 it can be seen that TLB1 (which has the benefit of a significantly higher Tg) is as effective as MTDATA at aiding hole injection.

1. A compound having the formula I 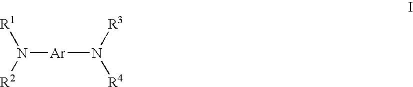
wherein Ar is an aromatic group selected from: 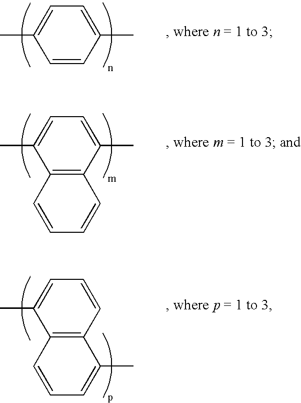
R¹ is a group selected from alkyl, alkenyl, cycloalkyl, cycloalkenyl, carbocyclic aryl optionally substituted by at least one group selected from halo, alkyl, cyano, nitro and cycloalkyl and an aromatic heterocyclic group optionally substituted by at least one group selected from halo, cyano, nitro, alkyl, cycloalkyl and aryl optionally substituted by at least one halo group;
R² is a fused bicyclic or tricyclic aromatic heterocyclic group selected from 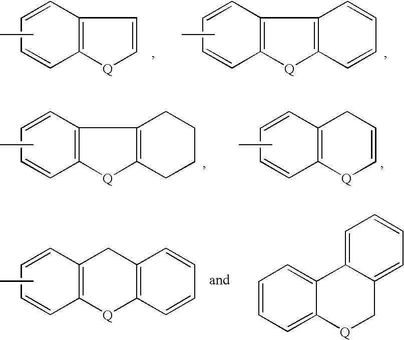
which heterocyclic group may, optionally, be substituted by at least one group selected from halo, cyano, nitro, alkyl, cycloalkyl and aryl optionally substituted by at least one halo and wherein Q is O, S or N—R⁵ where R⁵ is H, alkyl, cycloalkyl or aryl optionally substituted by at least one group selected from halo, alkyl, cyano or nitro.
R³ is a group selected from alkyl, alkenyl, cycloalkyl, cycloalkenyl, carbocyclic aryl optionally substituted by at least one group selected from halo, alkyl, cyano, nitro and cycloalkyl, and an aromatic heterocyclic group optionally substituted by at least one group selected from halo, cyano, nitro, alkyl, cycloalkyl and aryl optionally substituted by at least one halo; and
R⁴ is a group selected from carbocyclic aryl optionally substituted by at least one group selected from halo, cyano, nitro and alkyl, and aromatic heterocyclic optionally substituted by at least one group selected from halo, cyano, nitro, alkyl, aryl optionally substituted by at least one halo, and cycloalkyl.

2. A compound according to claim 1, wherein R² in formula I is an aromatic heterocyclic group selected from 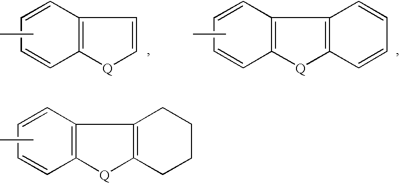
which heterocyclic group may, optionally be substituted by at least one group selected from halo, cyano, nitro, alkyl, cycloalkyl and aryl optionally substituted by at least one halo and wherein Q is O, S or N—R⁵ where R⁵ is H, alkyl, cycloalkyl or aryl optionally substituted by at least one group selected from halo, alkyl, cyano or nitro.

3. A compound according to claim 2, wherein R² is in the group 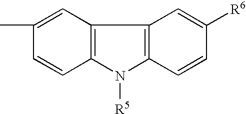
wherein R⁵ is as defined in claim 2 and R⁶ is H, halo, cyano, nitro, alkyl, cycloalkyl or aryl optionally substituted by at least one halo.

4. A compound according to any one of claims 1 to 3, wherein the groups R¹ and R³ in the formula I are each, independently, selected from phenyl, naphthyl and quinolyl.

5. A compound according to any one of claims 1 to 4, wherein R⁴ in formula I is phenyl, naphthyl or an aromatic heterocyclic group selected from 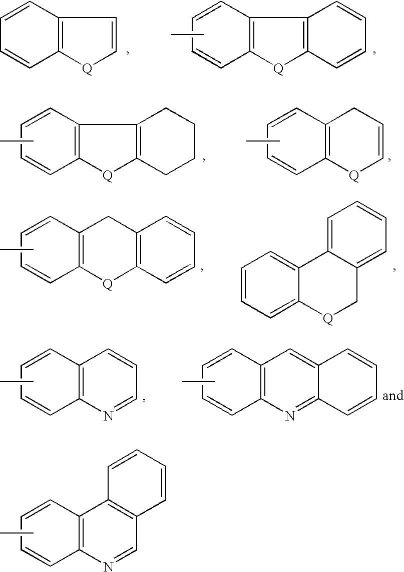
which heterocyclic group may, optionally, be substituted by at least one group selected from halo, cyano, nitro, alkyl, cycloalkyl or aryl optionally substituted by at least one halo and wherein Q is O, S or N—R⁵ where R⁵ is H, alkyl, cycloalkyl or aryl optionally substituted by at least one halo.

6. A compound according to claim 5 wherein R⁴ is the group 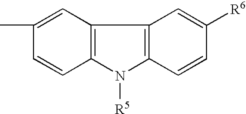
wherein R⁵ is as defined in claim 5 and R⁶ is H, halo, cyano, nitro, alkyl, cycloalkyl or aryl optionally substituted by at least one halo.

7. A compound according to claim 1, wherein, in formula I, Ar is the group 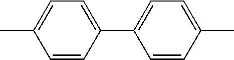
and R² and R⁴ are both a group of the formula

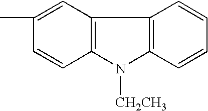

8. A compound according to claim 7, wherein, in the formula I, R¹ and R³ are both phenyl.

9. A compound according to claim 7, wherein, in formula I, R¹ and R³ are both 1-naphthyl.

10. A compound according to claim 7, wherein, in formula I, R¹ and R³ are both 6-quinolyl.

11. A process for producing a compound of the formula II 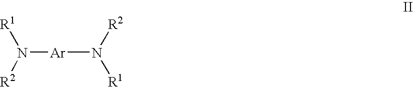
wherein R¹, R² and Ar are as defined in claim 1 which comprises the reacting a compound of the formula III
Br—Ar—Br III
with a compound of formula R¹—NH₂ to give a compound of the formula IV
R¹HN—Ar—NHR¹ IV
and then reacting the compound of the formula IV with a compound R²—X, where X is halogen.

12. An electroluminescent device comprising a compound according to any one of claims 1 to 9.

13. Use of a compound according to any one of claims 1 to 10 as a hole transporting material in an electroluminescent device.

14. Use of a compound according to any one of claims 1 to 10 as a hole injecting material in an electroluminescent device.
